(19) United States
(12) Patent Application Publication
(10) Pub. No.: US 2010/0330638 A1
(43) Pub. Date: Dec. 30, 2010

(54) Thermochemical Treatment of Lignocellulosics for the Production of Ethanol

(76) Inventors: Giovanna M. Aita, Baton Rouge, LA (US); Benito A. Stradi-Granados, San Jose (CR)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/866,994

(22) PCT Filed: Feb. 5, 2009
(86) PCT No.: PCT/US09/33173

§ 371 Date: Aug. 10, 2010

Related U.S. Application Data
(60) Provisional application No. 61/027,947, filed on Feb. 12, 2008.

(51) Int. Cl. C12P 7/06

(52) U.S. Cl. 435/161

(57) ABSTRACT

A method to process lignocellulosic biomass into ethanol under conditions of high biomass loading is disclosed. Pretreatment of biomass was conducted at a high concentration of solids but with a relatively low concentration of ammonia relative to the dry weight of biomass. The pretreated biomass was washed to remove inhibitors and to minimize the carry-over of the inhibitors to the subsequent steps of saccharification and fermentation. The pretreated-washed biomass is ground at some point prior to saccharification. Enzymes are added to allow saccharification and biomass liquification. More solids are added in a fed-batch manner as saccharification proceeds to ultimately obtain fermentation of a high-biomass concentration and get a higher ethanol titer. The amount of solids added in the fed-batch is such that the process achieves optimum hydrolysis to sugars by the saccharification enzymes.

(In countries other than the United States:) The benefit of the 12 Feb. 2008 filing date of U.S. provisional patent application 61/027,947 is claimed under applicable treaties and conventions. (In the United States:) The benefit of the 12 Feb. 2008 filing date of U.S. provisional patent application 61/027,947 is claimed under 35 U.S.C. §119(e).

This invention was made with the United States government support under contracts No. DE-FG36-04G014236 and DE-FG36-04G085007 awarded by the Department of Energy. The United States government has certain rights in this invention.

### TECHNICAL FIELD

This invention involves a procedure for the production of ethanol from lignocellulosics, for example, sugarcane bagasse, under conditions which allow high-solids loading and low-ammonia concentration.

### BACKGROUND ART

Sugarcane Bagasse

Sugarcane bagasse is a lignocellulosic material that on a mass basis contains 37%-43% cellulose, 20%-27% lignin and 18%-25% hemicellulose with the balance made-up by extractables and ash (NIST, 2001). Traditionally, ethanol comes from the fermentation of sugarcane juice. The biomass leftover after the extraction of sugar from sugarcane is called bagasse, and this lignocellulosic material is generally burned in boilers at the sugar mills to generate steam.

Use of sugarcane bagasse for ethanol requires four steps: (1) Pretreatment, (2) Washing, (3) Saccharification and fermentation, and (4) Distillation. The objective of the pretreatment is to render the cellulose portion in biomass available for hydrolyzation by enzymes and posterior fermentation with a biological agent, yeast in this case. There are numerous pretreatment methods or combinations of pretreatments available: physical (i.e. mechanical sheering, pyrolysis, freeze/thaw cycles and radiation) (Braemar Energy Ventures, 2007); thermochemical such as acid catalyzed (i.e. sulfuric acid, nitric acid, sulfur dioxide) (Cuzens and Miller, 1997), base catalyzed (ammonia, lime, sodium hydroxide, ammonia fiber explosion (AFEX)) (Gould, 1983; Kim and Holtzapple, 2006; Dale et. al., 1996; Kim and Lee, 2006), solvent-assisted (i.e. organosolv), chemical-based (i.e., peroxide and wet oxidation), non-catalyzed (i.e. high-temperature and near-supercritical water, steam explosion) and biologically assisted processes (i.e., both microbial and enzymatic processes) (Braemar Energy Ventures, 2007; Mok and Antal, 1992).

The pretreatment using dilute acid produces a liquid stream that contains a number of saccharification and fermentation inhibitors (Saha et al., 2005). Among these agents, there are furfural, 5-hydroxy-methyl furfural and acetic acid, which are compounds known to inhibit the function of enzymes and yeast. Alkali-based processes are known to be effective in the delignification of biomass particularly in pulping processes used in the paper industry; however, cost may prove prohibitory. Lime treatment is a low temperature process and requires leaving the biomass soaked into a lime solution for an extended period of time. The concentration of lime has to be sufficiently high to prevent the growth of bacteria while the biomass is delignified. Ammonia Fiber Explosion (AFEX) uses concentrated ammonia gas to treat the biomass; i.e., no liquid stream is used. AFEX is claimed to be highly effective with corn stover. Permeation of ammonia gas into more recalcitrant feedstocks (e.g., sugarcane bagasse) requires more severe conditions than those used for corn stover. Concentrated ammonium hydroxide has been used as a reactant in the pretreatment of biomass. However, the use of concentrated ammonia solution, like in the AFEX case, results in a more chemically hazardous operation. Pretreatment of biomass with high-temperature water has been pioneered by a number of researchers. High temperature water and near-supercritical water have dielectric constants and densities that differ significantly from that of liquid water at room temperature (Savage, 1999). The solubility of cellulose increases as both water temperature and pressure increases. In the near critical region, cellulose is totally soluble in water. Furthermore, hemicelluloses from bagasse can be completely extracted from biomass using hot water. This is a significant finding because in the absence of acid, a hot water process can carry out the same function as the dilute acid process.

### DISCLOSURE OF INVENTION

We have discovered a method to process lignocellulosic biomass into ethanol under conditions of high biomass loading. The process can involve the following steps: (1) Pretreatment, (2) Washing, (3) Grinding, (4) Pressing, (5) Separate initial saccharification with later fermentation (SHF), (6) Simultaneous saccharification and fermentation (SSF), and (7) Distillation (FIG. 1). Pretreatment of biomass was conducted at a high concentration of solids but with a relatively low concentration of ammonia relative to the dry weight of biomass. Once the biomass has been pretreated, it is washed to minimize the carry-over of the inhibitors to either the SHF or SSF step. The pretreated-washed biomass is ground at some point prior to saccharification. Enzymes are added to allow biomass liquification and saccharification. The simultaneous saccharification and fermentation step starts with the addition of yeast and a defined amount of solids. More solids are added in a fed-batch manner as the saccharification proceeds to obtain fermentation of a high biomass and get a higher ethanol titer. The amount of solids added in the fed-batch is such that it optimizes hydrolyzation of biomass to sugars.

The method comprises the following steps:
(a) A pretreatment in which biomass, water, and concentrated ammonium hydroxide solution are mixed in sufficient proportions to make cellulose available to enzymatic breakdown.
(b) The mixture of biomass, water, and concentrated ammonium hydroxide is heated to delignify and soften the biomass and to make the carbohydrates available for saccharification.
(c) The mixture from step (b) is washed and pressed to minimize the water and inhibitors remaining in the mixture.
(d) The mixture after step (c) is ground to increase surface area during enzyme saccharification. This grinding step could also be conducted prior to the dilute ammonium hydroxide pretreatment or prior to washing.
(e) The ground mixture is brought into contact with hydrolytic enzymes for saccharification to release fermentable sugars.
(f) The liquification of the mixture as a result of enzyme saccharification allows for the handling of high solids. The range of the amount of solids that could be handled for any one batch was determined by assaying the activity of the hydrolytic enzymes under different solids loading in the laboratory.
(g) The slurry composed of treated biomass and fermentable sugars is then mixed with a yeast culture in sufficient concentration to proceed with ethanol production.

Although developed for sugarcane bagasse, this technology fills the current need for a robust process capable of processing biomass from different sources to couple ethanol production to a commercial petroleum refining operation. Examples of other biomass that could be used with some minor modifications of operating procedures include corn stover, switchgrass, waste paper, corn grain, corn cobs, corn husks, wheat straw, hay, barley straw, rice straw, sugar cane bagasse, other grasses, sorghum, soy components, trees, branches, roots, leaves, wood chips, sawdust, shrubs, bush, and combinations thereof.

### BRIEF DESCRIPTION OF DRAWINGS

FIG. 1 illustrates a flowchart of one embodiment of the process for the production of ethanol from sugarcare bagasse.

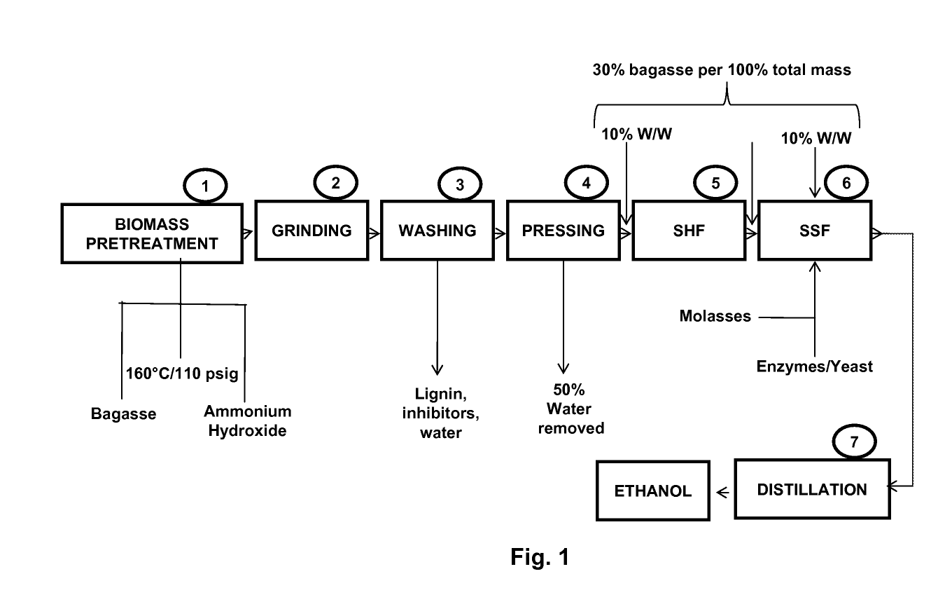

FIG. 2 illustrates a flowchart of ethanol production from lignocellulosic biomass using one embodiment of the invention, “Process I.” This embodiment of the invention depicts seven steps: (1) biomass pretreatment with dilute ammonium hydroxide; (2) washing of biomass to remove inhibitors; (3) grinding of biomass to increase surface area for enzyme saccharification; (4) pressing of pretreated and ground biomass to reduce moisture; (5) addition of enzymes during the initial separate hydrolysis for saccharification and later fermentation (SHF); (6) addition of yeast during simultaneous saccharification and fermentation (SSF); and (7) ethanol distillation. In this embodiment, steps (5) and (6) occur in the same reactor.

FIG. 3 illustrates a flowchart of ethanol production from lignocellulosic biomass using a second embodiment of the invention, “Process II.” This embodiment also depicts seven steps: (1) biomass pretreatment with dilute ammonium hydroxide; (2) washing of biomass to remove inhibitors; (3) grinding of biomass to increase surface area for enzyme saccharification; (4) pressing of pretreated and ground biomass to reduce moisture; (5) addition of enzymes during an initial separate hydrolysis and later fermentation (SHF); (6) addition of yeast during simultaneous saccharification and fermentation (SSF); and (7) ethanol distillation. In this embodiment, step (5) occurs in the same reactor as pretreatment to allow biomass liquification. The contents are then sent to a fermentor (a second reactor) to continue with saccharification and start the fermentation (SSF) process.

FIG. 4 illustrates the equipment for Process II: (A) Reactor used during biomass pretreatment and separate hydrolysis and fermentation (SHF); (B) Double-walled stainless steel cylinder attached to the bottom of the reactor post pretreatment for the washing and removal of inhibitors; (C) Flexible stainless steel hose fitted to a valve that controls the flow of partially liquefied biomass to the fermentor post SHF; and (D) Fermentor where liquefied biomass is collected in batches for the production of ethanol during simultaneous saccharification and fermentation (SSF).

FIG. 5 illustrates the enzymatic cellulose conversion to glucose at several concentrations of glucan loadings, reported after 48 h for 1% and 10% glucan loading, and after 72 h for 30% glucan loading.

FIG. 6 illustrates the amount of ethanol produced from sugarcane bagasse by Process I and Process II, as compared to the production from a cellulose standard (Avicel).

### MODES FOR CARRYING OUT THE INVENTION

### Definitions

In the specification and the claims, unless otherwise clearly indicated by context, the following terms are used with the definitions as indicated:

The term “biomass” refers to plant material which is primarily comprised of one or several of the following components: cellulose, hemicellulose, starch, oligosaccharides, and lignin.

The term “lignocellulosic” refers to biomass that contains cellulose, hemicellulose, and lignin. Examples of lignocellulosic biomass useful in this technology include corn stover, switchgrass, waste paper, corn grain, corn cobs, corn husks, wheat, wheat straw, hay, barley, barley straw, rice straw, sugar cane bagasse, other grasses, sorghum, soy components, trees, branches roots, leaves, wood chips, sawdust, shrubs, bush, and combinations thereof.

The term “pretreatment” refers to a process (i.e. thermochemical, physical, biological or combinations of the above) by which the biomass structure is distorted to make both cellulose and hemicellulose available for saccharification and fermentation.

The term “saccharification” refers to the process of converting complex carbohydrates, such as starch, cellulose or hemicellulose, into fermentable, monomeric sugars such as glucose and xylose. This process is essentially one of hydrolysis (use of water to breakdown a compound) and is often accomplished by the use of enzymes or acids.

The term “fermentation” typically refers to the conversion of sugars, particularly glucose, to an alcohol or ethanol by microorganisms. Examples of fermentation microorganisms include wild-type or modified organisms selected from the group consisting of Saccharomyces cerevisiae, Escherichia coli, Klebsiella spp., Zymomonas mobilis, Clostridium acetobutylicum, Bacillus stearothermophilus, and Pichia stipitis.

The term “separate hydrolysis and fermentation (SHF)” refers to the addition of a microorganism after partial biomass saccharification by enzymes has been completed.

The term “simultaneous saccharification and fermentation (SSF)” refers to the operation by which the sugars produced by saccharification are immediately consumed by microorganisms for fermentation. Saccharification by enzymes and fermentation by microorganisms are occurring at the same time.

The term “batch” refers to a process carried out in a single vessel to and from which essentially no mass is added or removed. The term “fed-batch” refers to the operation of adding substrate to a process that is ongoing, and that was initiated in a batch mode. For example, an initial saccharification step may begin in a batch mode in which a set amount of pretreated biomass and enzymes are placed in the reactor. After a specified period of time, an additional amount of biomass is added to the mixture in the same reactor vessel. This addition of new biomass constitutes a “fed-batch” operation.

In the following description, specific values are meant to indicate close to the optimum values applicable to sugarcane bagasse. However, small deviations of ±10% in temperature or pressure are not significant variations of the method. The procedure is robust and is meant for a range of biomass materials. Examples of lignocellulosic biomass useful in this technology include corn stover, switchgrass, waste paper, corn grain, corn cobs, corn husks, wheat, wheat straw, hay, barley, barley straw, rice straw, sugar cane bagasse, other grasses, sorghum, soy components, trees, branches roots, leaves, wood chips, sawdust, shrubs, bush, and combinations thereof.

The invention is a method to produce ethanol from sugarcane bagasse, which can include the following steps: (1) Pretreatment, (2) Washing, (3) Grinding, (4) Pressing (5) Separate initial hydrolysis and later fermentation (SHF), (6) Simultaneous saccharification and fermentation (SSF), and (7) Distillation. Pretreatment breaks the biomass structure composed mostly of cellulose, hemicellulose, and lignin. Pretreatment is preferably done with diluted ammonia. A stock solution of ammonium hydroxide with an ammonia concentration of about 28-30 g NH₃ in 100 g solution was purchased (Mallinckrodt Chemicals, New Jersey). From this solution, about 0.5 g was added to the reactor (about 0.14 g NH₃ per gram dry biomass). The final weight proportion of biomass, water, and pure ammonia was about 1.0:8.0:0.14, respectively. Higher ammonia concentrations resulted in undesirable high levels of ammonia residue in the pretreated biomass, which would require special equipment to handle safely. An important step in the development of the current process was to titrate the amount of ammonia used to be just enough to pretreat the biomass with minimal residual amounts of ammonia after the pretreatment. The cellulose becomes available for saccharification as a result of the pretreatment. Washing and pressing removes inhibitors generated during the pretreatment step. Grinding increases the surface area of the biomass for saccharification, and can be done either before or after pretreatment. Saccharification is performed by a mixture of enzymes that hydrolyze the cellulose into glucose. Finally, in the fermentation step, glucose is consumed by yeast (Saccharomyces cerevisiae) under anaerobic conditions to generate alcohol. The initial concentration of solids is increased by feeding addition amounts of low-moisture treated biomass to the batch reactor. The final broth is distilled to an azeotropic mixture of ethanol and water. Absolute ethanol (200% proof) requires an absorbent material or equivalent technology to remove the water remaining in the azeotropic mixture coming out of the distillation unit.

Described herein are two embodiments of this technology—Process I and Process II. The difference between the two processes is that Process I describes the use of a single reactor unit for pretreatment, SHF and SSF, and Process II uses the same reactor for only pretreatment and SHF. Process II takes advantage of liquification by the hydrolytic enzymes during saccharification to flow the contents into a second vessel for fermentation where SSF takes place. Both processes can accommodate high solids loading using the fed-batch approach described above. For example, 30% total solids were loaded using three loadings of 10% each.

### Example 1

### Sugarcane Bagasse Composition Analysis

Biomass composition (cellulose, hemicelluloses and lignin) was calculated before and after pretreatment following National Renewable Energy Laboratory's (NREL) Analytical Laboratory Procedure (LAP 002, 2006) for the determination of carbohydrates and lignin in biomass (Table 1). Composition is calculated in mass percent units, i.e., grams per 100 grams dry biomass. The biomass was dried for 24 h at 110° C. in an oven prior to analyses. Sugars were analyzed (i.e., glucose, cellobiose, mannose, arabinose and xylose) by using an Aminex® HPX-87P, 300 mm×7.8 mm column. Samples were run for 22 min in water at 80° C. All sugar standards were purchased from Sigma Chemical Co. (St. Louis, Mo.).

TABLE 1
Composition of untreated, dilute ammonium
hydroxide-treated, and water-treated
sugarcane bagasse.

| | Sugarcane Bagasse | | |
|---|---|---|---|
| Biomass Constituent | | | Dilute Ammonium |
| (g/100 g dry biomass) | Untreated | Water† | Hydroxide |
| Glucan (Cellulose) | 38.4 | 60.5 | 63.6 |
| Xylan (Hemicellulose) | 24.1 | 13.3 | 24 |
| Lignin | 25 | 24.3 | 21.1 |

† = Sugarcane bagasse was treated with water only at 160° C. for 1 hour as control.

TABLE 2
Percent glucan present at each percent loading of treated
sugarcane bagasse (water and ammonium hydroxide) and
pure cellulose (Avicel and SOLKA-FLOC ®).

| | | Percent Solids Loading* | |
|---|---|---|---|
| | | (g dry biomass/100 g total mass) | |
| Material | 1% | 10% | 30% |
| Avicel | 1 | 10 | 30 |
| SOLKA-FLOC ® | 1 | 10 | 30 |
| Bagasse (Water) | 0.6 | 6.1 | 18.2 |
| Bagasse (Dilute | 0.6 | 5.7 | 17 |
| Ammonium Hydroxide) | | | |

* = Values are given as percent glucan (g glucan/100 g dry biomass) at each percent solid loading (g dry biomass/100 g total mass).

### Example 2

### Determination of Most Effective Solids Loading

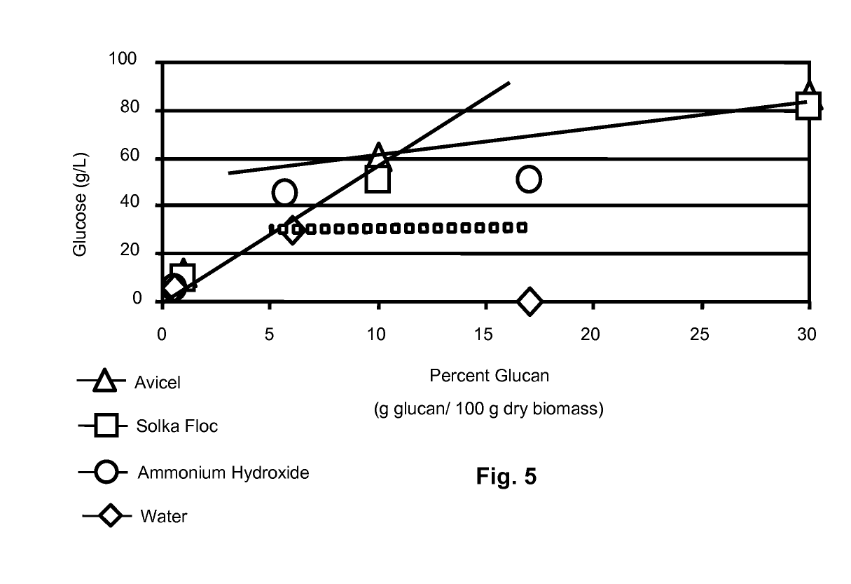

The amount of glucose (g/L) released after enzyme saccharification of biomass at high percent glucan loadings is presented in FIG. 5. SOLKA-FLOC® and Avicel (pure cellulose) were hydrolyzed to 10 g/L, 50-60 g/L, and 80-85 g/L of glucose at 1%, 10% and 30% glucan loadings, respectively. These values represent the maximum amount of glucose that can be obtained under current experimental conditions. Glucose levels for the dilute ammonium-hydroxide treated biomass were 5 g/L, 45 g/L and 50-55 g/L at 0.6%, 5.7% and 17% glucan loadings, respectively. These values represent at least 85% theoretical cellulose conversion. Glucose levels for water-treated biomass were lower.

In FIG. 5, Lines (1) and (2) provide the maximum limits for saccharification that are given by the pure cellulose standards (Avicel and SOLKA-FLOC®). It is not possible to obtain more glucose out of a pretreated biomass sample than that obtained when using pure cellulose. The crossing point of lines (1) and (2) determines the maximum of solid loadings (percent glucan) while achieving maximum saccharification (g glucose/L solution). Consequently, in the current protocol, the saccharification and fermentation was initiated at a solids concentration below 10% glucan (5.7 glucan loading) to achieve maximum saccharification in the shortest possible time. Once the glucose is consumed by the yeast during the simultaneous saccharification and fermentation, then more biomass was added to the batch reactor. This created a fed-batch operation that maintained the reactor operating in the region of highest saccharification rate. Beyond the intersection of lines (1) and (2), the rate of saccharification for biomass is much lower than those expected from using only cellulose. This determination of saccharification efficiency to determine the most effective solids loading has not been published before. This determination provides a fail-safe operating range for use in batch and fed-batch operations to maximize the saccharification of biomass, glucose generation, and ethanol production.

### Example 3

### Pretreatment, Washing, Grinding and Pressing

Pretreatment. Biomass, water and ammonium hydroxide are used in mass proportions of about 1:8:0.14. The sugarcane bagasse was used without drying and contained about 24% moisture. Water was added to the biomass in sufficient quantity to prepare a slurry. The stock ammonium hydroxide solution was weighed into a pressurized stainless steel container and mixed with water. The ammonium hydroxide and water mixture in the pressurized stainless steel container was then emptied into the reactor with the biomass by pressurizing the container with air. The reactor was a cylindrical, high-pressure vessel from Ohio Valley Steel (Ohio) built with INCONEL® alloy, with a volume of 22.1 liters (at 22° C.), and equipped with a jacket for heating and cooling. The reactor had pressure and temperature gauges for monitoring and controlling operating conditions. Top and bottom ports were fitted with quick-disconnect ports for the washing step. The mixture was agitated through a system of gears half-way through the vessel's height. The reactor rotated slowly around its shorter axis in a tumbling motion at about 2 revolutions per minute. Heating was provided by high pressure steam at a temperature of 180° C. that flowed through the reactor jacket. The reactor had an automatic glove valve at the bottom that could be opened to discharge the biomass from the reactor.

In a normal run, about 1.9 kg (dry weight) sugarcane bagasse was loaded into the reactor along with 12.2 kg water. The reaction vessel was closed, and the gauges checked for nominal operation. Additional water (3 kg) was mixed with 0.95 kg stock solution of ammonium hydroxide (28%-30% w/w) in a pressurized stainless steel container, and the diluted solution added to the reactor. The ammonium hydroxide and water mixture was loaded into the reactor last as a safety precaution when the reactor was already closed, avoiding any ammonia escaping from the reactor mixture. Heating was then initiated with full steam pressure in the jacket. A heating period of about 20 min was needed for the reactor to reach an internal temperature of about 160° C. Once the internal temperature was reached, the contents of the reactor were rotated continuously for about 1 h. In an earlier experiment, the reactor was heated to 120° C. and rotated continuously for about 2 h. After an hour of pretreatment, the reactor was cooled down to about 50° C.-80° C., and the contents discharged into a custom-made double-walled stainless steel cylinder specially design to wash the biomass.

Washing. The washing step was carried out in a specially design stainless steel double-walled cylinder. Once the pretreatment ended, the double-walled cylinder was coupled to the automatic valve at the bottom of the reactor prior to discharging the biomass. The cylinder was about 27.5 inches tall and 7.25 inches in diameter. The walls were made of two superposed stainless-steel meshes. The inner mesh was gauge 20 (about 0.02 inches in diameter) that retains the treated biomass but allows liquid to flow through. The outer mesh was gauge 8 (about 0.08 inches in diameter). The primary objective of the finer mesh was to retain the solids, while the coarser one provided mechanical support against the impact from the biomass discharge under pressure. Approximately, 20 kg condensed steam (collected in a stainless steel container) were pumped over the top of the reactor and through the double-walled cylinder containing the pretreated sugarcane bagasse. The pumped water was then re-circulated for 10 min to wash away inhibitors and any residual ammonium hydroxide solution.

Grinding. In the preferred embodiment, the grinding step occurs after the pretreatment. However, grinding could occur prior to pretreatment or at any time prior to addition of the hydrolytic enzymes. The washed biomass was taken out of the cylinder and ground using a standard meat grinder (¼-inch hole, 2.25-diameter grinding plate, 1 HP (horsepower)). Grinding the biomass prior to pretreatment requires a higher energy for grinding since raw sugarcane bagasse does not grind as easily as bagasse after dilute ammonia pretreatment. After pretreatment (about 4% lignin has been removed), the bagasse was softer and the grinding process was more efficient and economical. Most of the unground bagasse (about 73%) had a particle size of greater than 0.0331 in; and only a small amount (about 2%) had a particle size of less than about 0.0139. After grinding, about 61% of the ground bagasse has a particle size between 0.0331 and 0.0139 in., 29% has a size less than about 0.0139 in; and only about 11% has a particle size greater than 0.0331 in. Particle size reduction whether taking place before or after pretreatment helps increase the surface area of biomass for attack by the hydrolytic enzymes, thus improving enzyme saccharification.

Pressing. Pressing of the pretreated-ground bagasse prior to saccharification was done to remove excess water along with dissolved inhibitors of enzyme hydrolysis that were generated during the dilute ammonia pretreatment. The moisture content of pretreated-washed-ground sugarcane bagasse was lowered by about 50% using a standard sugar mill which squeezes the water out of the biomass. The amount of water remaining in the pressed biomass allowed handling of high solids which resulted in higher ethanol concentrations. The final pH of the pressed bagasse ranged from about pH 6.8 to about pH 7.0.

### Example 4

### Separate Hydrolysis and Fermentation

The next step (Separate Hydrolysis and Fermentation or SHF) was done in the same reactor used for the pretreatment of sugarcane bagasse as described above. The pretreated, pressed biomass with 50% moisture content was sterilized at about 121° C. for about 30 min in the reactor. After sterilization, the internal temperature of the reactor was lowered to a temperature from about 55° C. to about 33° C. (optimal temperature for enzymes). Then the following commercially available hydrolytic enzymes were added: from about 15 to about 64 Cellobiose Unit (CBU)/g glucan of glucan b-glucosidase (Novozyme 188) (a cellobiose from Aspergillus niger; Novozymes; Davis, Calif.) and from about 30 to about 60 Spezyme CP (Genencor Inc.; Rochester, N.Y.) at concentrations ranging from about 15 to about 64 Cellobrose Unit (CBU) Filter Paper Units (FPU) per gram of glucan. Then the mixture was complemented with yeast extract (1% w/w), soy peptone (2% w/w), water and citrate buffer (0.5 M) to start saccharification. The final concentration in the reactor was about 10 g of dry solids (sugarcane bagasse) per 100 g of total mass. Saccharification was allowed to proceed anywhere from about 2 h up to about 24 h. Liquification of pretreated bagasse was seen within 2 h of enzyme addition. Fermentable sugars (i.e., glucose, cellobiose, mannose, arabinose, and xylose) were analyzed using an Aminex® HPX-87K, 300 mm×7.8 mm column, as described above. Samples were run for 25 min in 0.01 M H₂SO₄ solution at 80° C.

### Example 5

### Simultaneous Saccharification and Fermentation

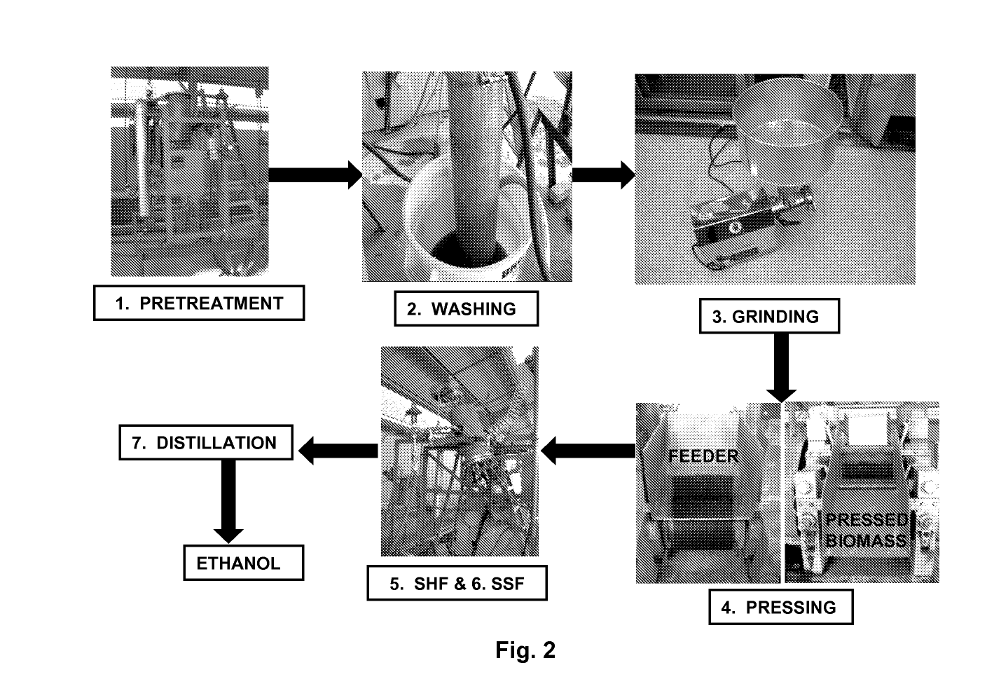

During Process I (FIG. 2), upon liquification of pretreated bagasse (10% dry solids) at elevated temperature, additional pressed, pretreated biomass was added to the reactor (as fed-batch) to increase the solids concentration to 30 g solids per 100 g total mass (both solids and liquids), followed by the addition of molasses (5% w/w) and Saccharomyces cerevisiae (ATCC 200062; American Type Culture Collection, Manassas, Va.) to a final concentration of 10⁷ CFU per milliliter. The temperature in the reactor was maintained at 33° C. once the yeast and molasses were added. The yeast was prepared as described in NREL Laboratory Analytical Procedure LAP-008 (NREL, 1995). The fed-batch process was used to increase the concentration of biomass in the reactor. The fibrous nature of the bagasse made it very difficult to feed the reactor with high solids during SHF unless it was done as a fed-batch. Once saccharification began, the biomass liquified; thus making the material better suited for transport. Additionally, product inhibition becomes an issue at high solids loading. The reactor was fed in installments through the bottom valve opening of the reactor as a way to increase the biomass concentration without the use of additional water as a transportation fluid and as a way to help prevent substrate inhibition. Feeding in installments together with the consumption of sugars by the yeast permitted the control of free sugar concentration to below inhibitory levels. The controlled addition of biomass increased the amount of sugars formed by saccharification and thus increased the overall ethanol production.

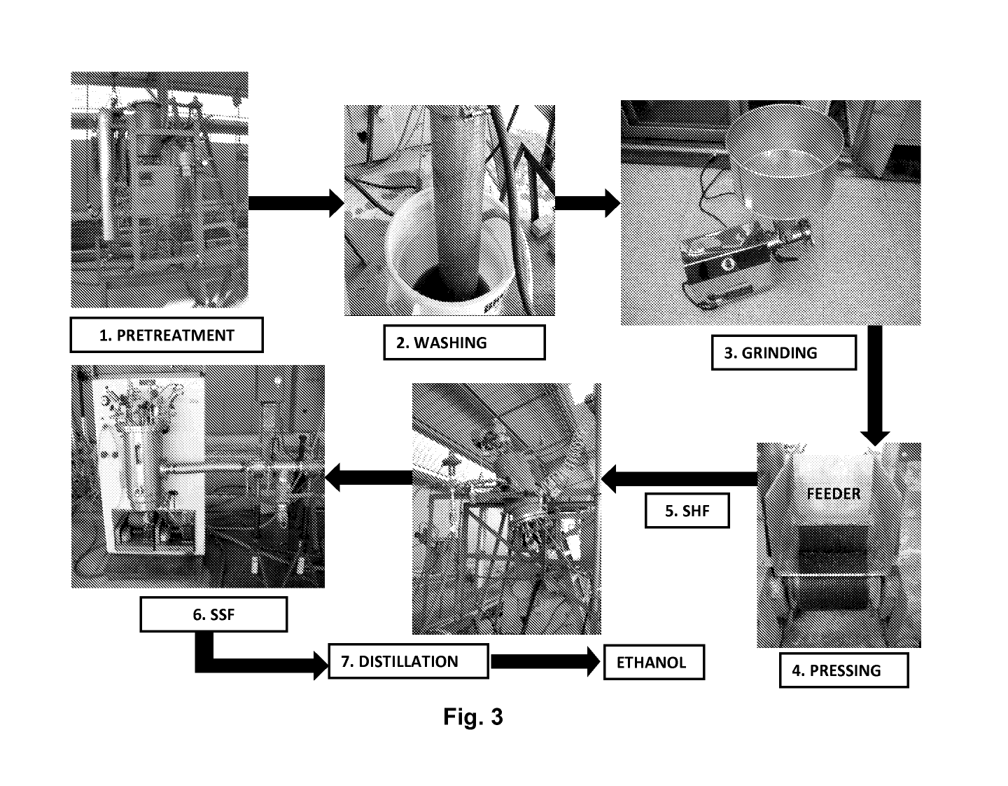

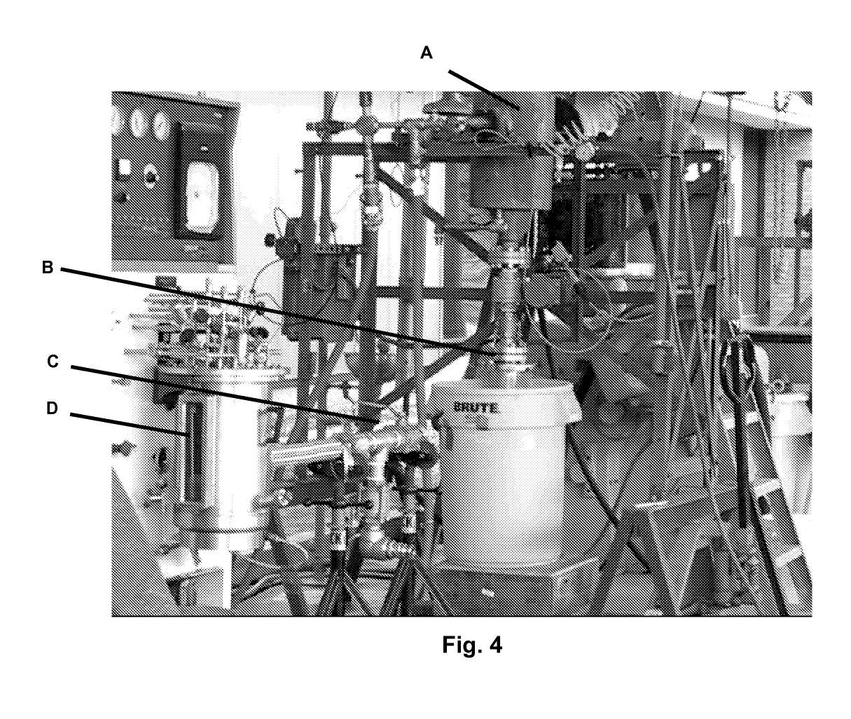

During Process II (FIGS. 3 and 4), the first batch (10% dry solid) of liquified, saccharified biomass (2 h to 24 h) was sent into a 28 L fermentor (Microferm, New Brunswick Scientific Co., Inc.) via flexible stainless steel tubing. One end of the tubing was connected to the bottom of the reactor and the other end to an opening on the side of the fermentor. The reactor was pressurized (about 20-30 psi), and the contents pressure-pushed into the fermentor. Yeast and molasses was either added to the fermentor or to the reactor before discharge. SHF was repeated with the remaining 20% dry solids, and the partially saccharified biomass was collected and added to the fermentor. In the final stage at about 10 h to about 48 h, the fermentor contained a mixture resulting from a 30% solids loading. SSF proceeded in the fermentor for additional 24 h to 48 h at about 33° C.

Fermentable sugars and alcohol were analyzed using an Aminex® HPX-87K, 300 mm×7.8 mm column. Samples were run for 25 min in 0.01 M H₂SO₄ solution at 80° C. Ethanol was also quantified by GC using a Supelco™, 60 m×0.32 m Carbowax capillary column for 15 min at 220° C.

### Example 6

### Ethanol Production and Distillation

Ethanol was produced from the fermentation of the monomeric sugars derived from the saccharification of lignocellulosic sugarcane bagasse. In Process I, ethanol was produced in the reactor by the addition of yeast with a molasses supplementation. In process II, yeast and molasses were added either to the reactor post-SHF after liquification had taken place, or to the fermentor after the liquified biomass was transferred. The type and amount of yeast are given above. After 48 h, the contents of the reactor (Process I) or fermentor (Process II) were taken into a clarifier where the solids were removed. The ethanol-water-broth mixture was distilled in a 22 plate distillation column, and the azeotropic ethanol-water mixture was further dried over calcium sulfate or other suitable desiccant.

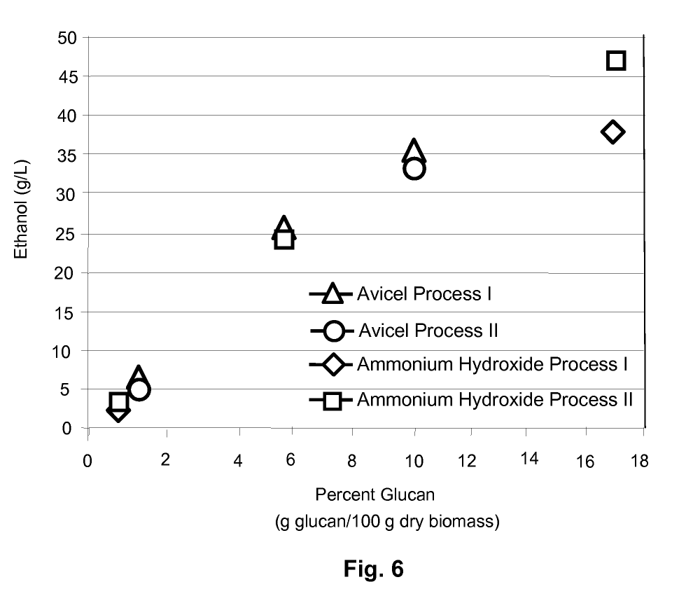

The combined addition of enzymes and yeast cells to minimize product (glucose) inhibition at high glucan loadings produced higher ethanol yields. FIG. 6 shows the amount of ethanol produced at the end of the two fermentation processes, as compared to the sugar controls. No significant difference in ethanol production from either process was observed. At 17% glucan (g glucan/100 g dry biomass), 38 g/L ethanol were obtained for process I compared to 46 g/L ethanol obtained for process II, without molasses supplementation.

The sugar composition of molasses used as carbon source and nutrient supplement during SSF studies is presented in Table 3.

TABLE 3
Blackstrap molasses sugar composition.

| Sugar | Percent (g/100 g molasses) |
|---|---|
| Sucrose | 25.3 |
| Glucose | 8.1 |
| Fructose | 9.5 |

Ethanol production at 17% glucan (g glucan/100 g dry biomass) increased when the biomass mixture was supplemented with molasses (Table 4). Unfermented sugars were observed with 10% (w/w) ethanol in the fermentation broth, an indication that the yeast (Saccharomyces cerevisiae ATCC 200062) had reached its alcohol tolerance level.

TABLE 4
Ethanol yields by media supplementation with blackstrap molasses.

| | Molasses (g/ | Glucose# | Ethanol (g/ |
|---|---|---|---|
| | 100 g total | (g/liter of | liter of |
| | fermentation | fermentation | fermentation |
| Treatment | solution) | liquid) | liquid) |
| Ammonium Hydroxide† | 0 | 0.1 | 3.8 |
| Ammonium Hydroxide† | 5 | 0.2 | 5.6 |
| Ammonium Hydroxide† | 15 | 4.6 | 13.5 |
| Ammonium Hydroxide* | 0 | 0.1 | 3.4 |
| Ammonium Hydroxide* | 5 | 0.2 | 5.1 |
| Ammonium Hydroxide* | 15 | 1.6 | 10.4 |

# = Grams of glucose per liter of fermentation liquid at the end of SSF
† = Enzymes added at a concentration of 60 FPU/g glucan (Spezyme CP) and 30 CBU/g glucan (Novozyme 188)
* = Enzymes added at a concentration of 30 FPU/g glucan (Spezyme CP) and 15 CBU/g glucan (Novozyme 188)

### REFERENCES

1. Braemar Energy Ventures (2007). A Financial Perspective on Bioenergy: Biomass Research Development Intake. http://www.brdisolutions.com/Site%20Docs/TAC%20Meeting%20September%2010-11,%202007/Cellulosic%20Ethanol%20-BRDI5.pdf (accessed on Dec. 5, 2007).
2. Cuzens, J. C. and Miller, J. R. (1997). Acid Hydrolysis of Bagasse for Ethanol Production. Renewable Energy, 10: 285-90.
3. Dale, B. E., Leon, C. K., Pham, T. K., Esquivel, V. M., Rios, I. and Latimer, V. M. (1996). Hydrolysis of Lignocellulosics at Low Enzyme Levels: Application of the AFEX Process. Bioresource Technology, 56: 111-116.
4. Gould, J. M. (1984). Alkaline Peroxidase Delignification of Agricultural Residues to Enhance Enzymatic Saccharification. Biotechnology and Bioengineering, 26: 46-52.
5. Kim, S. and Holtzapple, M. T. (2006). Delignification Kinetics of Corn Stover in a Lime Pretreatment. Bioresource Technology, 97: 778-785.
6. Kim, T. H. and Lee, Y. Y. (2006). Fractionation of Corn Stover by Hot-Water and Aqueous Ammonia Treatment. Bioresource Technology, 97: 224-232.
7. Mok, W. S-L. and Antal, J. M. (1992). Uncatalyzed Solvolysis of Whole Biomass Hemicellulose by Hot Compressed Liquid Water. Industrial Engineering Chemical Research, 31: 1157-1161.
8. National Institute of Standards & Technology (NIST) (2005). Determination of Structural Carbohydrates and Lignin in Biomass. http://devafdc.nrel.gov/pdfs/9572.pdf (accessed on February, 2006).
9. National Institute of Standards & Technology (NIST) (2001). Report of Investigation: Whole Biomass Feedstocks. https://srmors.nist.gov/certificates8491)pdf?CFID=13248108&CFTOKEN=bbdc0b9924eb9956-AF1B8FC0-03FA-BCF3050F11A2224 895D3&jsessionid=b43067e476323b534b22 (accessed on January, 2008)
10. National Institute of Standards & Technology (NIST) (1995). LAP 008: SSF Experimental Protocols: Lignocellulosic Biomass Hydrolysis and Fermentation. http://cobweb.ecn.purdue.edu/˜lorre/16/research/LAP-008.pdf (accessed on October, 2005).
11. Saha, B. C., Iten, L., Cotta, M. and Wu, Y. V. (2005). Dilute Acid Pretreatment, Enzymatic Saccharification and Fermentation of Wheat Straw to Ethanol. Process Biochemistry, 40: 3693-3700.
12. Savage, P. E. (1999). Organic Reactions in Supercritical Water. Chemical Reviews, 99: 603-621.

The complete disclosures of all references cited in this specification are hereby incorporated by reference. In the event of an otherwise irreconcilable conflict, however, the present specification shall control.

1. A method for producing ethanol from a lignocellulosic biomass using a fed-batch system, said method comprising the steps of:
a) Treating up to a 15% aqueous lignocellulosic biomass solution (wt dry biomass/wt total mass (biomass plus water)) with an aqueous dilute ammonium hydroxide solution to a temperature greater than 100° C. for a time sufficient to increase the surface area of the biomass to enzyme hydrolysis;
b) Washing the treated biomass with water;
c) Removing at least 40% of the water from the treated, washed biomass;
d) Contacting the de-watered biomass with a saccharification enzyme under conditions conducive to producing fermentable sugars;
e) Grinding the biomass after step (a) and prior to step (d);
f) Repeating steps (a) through (e) up to two times; and
g) Contacting the fermentable sugars with a fermentation microorganism to produce ethanol.

2. The method of claim 1, wherein the lignocellulosic biomass is selected from the group consisting of switchgrass, waste paper, corn grain, corn cobs, corn husks, corn stover, wheat, wheat straw, hay, barley, barley straw, rice straw, sugar cane bagasse, other grasses, sorghum, soy components, trees, branches roots, leaves, wood chips, sawdust, shrubs, bush, and combinations thereof.

3. The method of claim 1, wherein the lignocellulosic biomass is sugarcane bagasse.

4. The method of claim 1, wherein the ratio of lignocellulosic biomass to water to ammonium hydroxide by mass in step (a) is about 1.0:8.0:0.14.

5. The method of claim 1, wherein the amount of lignocellulosic biomass in step (a) is equal to or less than a calculated optimal amount of lignocellulosic biomass for saccharification by the saccharification enzyme of step (d).

6. The method of claim 1, wherein the temperature of step (a) is greater than about 120° C.

7. The method of claim 1, wherein the temperature of step (a) is from about 160° C. to about 220° C.

8. The method of claim 1, wherein the time for treatment of step (a) is about 1 hour.

9. The method of claim 1, wherein washing step (b) occurs in a double-walled cylinder.

10. The method of claim 1, wherein water removal in step (c) occurs by squeezing the water from the biomass.

11. The method of claim 1, wherein the saccharification enzyme of step (d) is a cellulase.

12. The method of claim 1, wherein the fermentation organism of step (f) is a wild-type, cultured, or modified organism selected from the group consisting of Saccharomyces cerevisiae, Escherichia coli, Klebsiella spp., Zymomonas mobilis, Clostridium acetobutylicum, Bacillus stearothermophilus, and Pichia stipitis.

13. The method of claim 1, wherein the fermentation organism of step (f) is Saccharomyces cerevisiae.

14. The method of claim 1, additionally comprising the step of adding an additional sugar source to the fermentable sugars in step (g).

15. The method of claim 14, wherein the additional sugar source is molasses.

16. The method of claim 1, additionally comprising dividing the product from step (g) into a fluid portion and a solid portion, and measuring the ethanol produced in the fluid portion.

17. The method of claim 16, wherein the produced ethanol is at least 40 g ethanol per liter fluid portion.

18. The method of claim 1, wherein steps (d) and (g) occur in the same reactor.

19. The method of claim 1, wherein steps (d) and (g) occur in separate reactors.

20. A method of producing ethanol from lignocellulosic biomass using a fed-batch system, wherein the amount of lignocellulosic biomass in a batch is determined by assaying for the amount of lignocellulosic biomass equal to or less than the amount that produces optimum hydrolysis by one or more saccharification enzymes.

21. The method of claim 20, wherein the optimal amount of lignocellulosic biomass is about 10 g of dry biomass per 100 g of total mass.
